(19) United States
(12) Patent Application Publication
(10) Pub. No.: US 2003/0092978 A1
(43) Pub. Date: May 15, 2003

(54) Flexible medical electrode

(76) Inventor: Richard J. Fisher III, Columbus, OH (US)

Correspondence Address: DAVID A. GREENLEE, P.O. BOX 340557, COLUMBUS, OH, 43234-0557, US

(21) Appl. No.: 10/008,798

(22) Filed: Nov. 13, 2001

(51) Int. Cl. A61B 5/04

(52) U.S. Cl. 600/391

(57) ABSTRACT

A flexible laminated medical electrode for use on a human body part comprises a backing layer of flexible, non-conductive material, a conductive layer of carbon-loaded vinyl covered with a conductive ink that is formed with a plurality of furcations extending from a central spine, and a hydrogel layer to provide an electrode having sufficient flexure to enable conformance to the contours of the body parts without adversely affecting electrical conductivity throughout the electrode. The medical electrode is made by cutting a sheet of conductive film to provide a continuous shape having a plurality of furcations extending from a central spine, inserting this sheet between a sheet of hydrogel on a backing liner and a sheet of flexible, non-conductive material having an adhesive coating on an inner side that is partially covered with a release liner, inserting the conductive film sheet between the sheet of hydrogel and the inner side of the sheet of non-conductive material to form a partial laminate, cutting the partial laminate into discrete electrodes, folding back the liner-covered portion of non-conductive material of each electrode to expose the central spine, removing the release liner, applying a lead wire along the central spine, and engaging the non-conductive material with the conductive film sheet to secure the lead wire to the central spine.

### BACKGROUND OF THE INVENTION

1. Technical Field

This invention relates to medical electrodes and, more particularly, to such an electrode that is sufficiently flexible to readily conform to human body parts while maintaining electrical continuity throughout the electrode.

2. Background Art

Medical electrodes are used on the skin of various body parts and must frequently be used on curved surfaces, both concave and convex. Thus, to achieve good conductivity over the extent of the electrode, it is necessary for the electrode to be flexible. Since millions of these electrodes are used annually, cost is a prime consideration.

Round or square solid electrodes of very thin, flexible material are in common use. However, these tend to crinkle or stretch if forced onto a curved surface. Also in common use are electrodes formed of a conductive mesh material. These tend to be cumbersome and suffer the same crinkling. They are also expensive.

Many electrodes are subject to exhibiting “edge bite” in which current leaks out of the periphery of the electrode and onto the skin surface.

The electrode disclosed in U.S. Pat. No. 4,736,752 comprises a laminated electrode having a conductive element in the form of a grid formed of a printed pattern of conductive ink and a mask layer to prevent edge bite. Although flexible, this electrode is subject to fracture of the fragile conductive element if stretched or bent.

It would be desirable to provide a medical electrode for human use that is flexible, inexpensive and immune from electrical discontinuities of the conductive element if bent or stretched.

### BRIEF SUMMARY OF THE INVENTION

It is an object of this invention to provide a medical electrode for human use that is flexible, inexpensive and immune from electrical discontinuities of the conductive element if bent or stretched.

In one aspect this invention features a flexible laminated medical electrode for use on a human body part, comprising a first backing layer of flexible, non-conductive material. A second conductive layer is a sheet of carbon-loaded vinyl covered with a conductive ink and formed with a plurality of furcations extending from a central spine that includes means for connecting the electrode to a power source. A third hydrogel layer completes this laminate, which provides sufficient flexure for the electrode to enable conformance to the contours of the body parts without adversely affecting electrical conductivity throughout the electrode.

Preferably, the conductive layer has at least four second conductive layer furcations which extend perpendicularly from opposite sides of the central spine and are spaced inwardly of the electrode periphery.

In another aspect, this invention comprises a method of making a flexible laminated medical electrode, comprising the steps of providing an elongated sheet of conductive film that is flood-coated with conductive ink, cutting said sheet to provide a continuous shape having a plurality of furcations extending from a central spine, providing an elongated sheet of hydrogel on a backing liner, providing an elongated sheet of flexible, non-conductive material having an adhesive coating on an inner side that is partially covered with a release liner so as to leave an exposed adhesive strip along one edge, inserting said conductive film sheet between the sheet of hydrogel and the inner side of the sheet of non-conductive material to form an elongated sheet of partial laminate, with the adhesive strip securing a portion of the non-conductive material to the conductive film, cutting the sheet of partial laminate into discrete partially laminated electrodes, folding back the liner-covered portion of non-conductive material of each electrode to expose said central spine, removing the release liner, applying a lead wire along the central spine, and engaging the non-conductive material with the conductive film sheet to secure the lead wire to the central spine and thereby form said flexible laminated medical electrode.

These and other objects and features of this invention will become more readily apparent upon reference to the following detailed description of a preferred embodiment, as illustrated in the accompanying drawings, in which:

### BRIEF DESCRIPTION OF THE DRAWINGS

FIG. 1 is a plan view of a flexible medical electrode according to this invention;

FIG. 2 is a plan view of a strip of conductive film used in this invention;

FIG. 3 is a side view the layers of material used in forming the electrode of this invention;

FIG. 4 is a plan view of the materials of FIG. 3 during processing, stamped in the form of individual electrodes before the removal of scrap and backing material;

FIG. 5 is an end view of the electrode if FIG. 4, showing the top backing layer peeled back to enable removal of the liner layer;

FIG. 6 is a view similar to FIG. 5, showing the electrode in final laminated form, after addition of a contact electrode; and

FIG. 7 is a plan view of another embodiment of a flexible medical electrode, according to this invention.

### DETAILED DESCRIPTION OF THE INVENTION

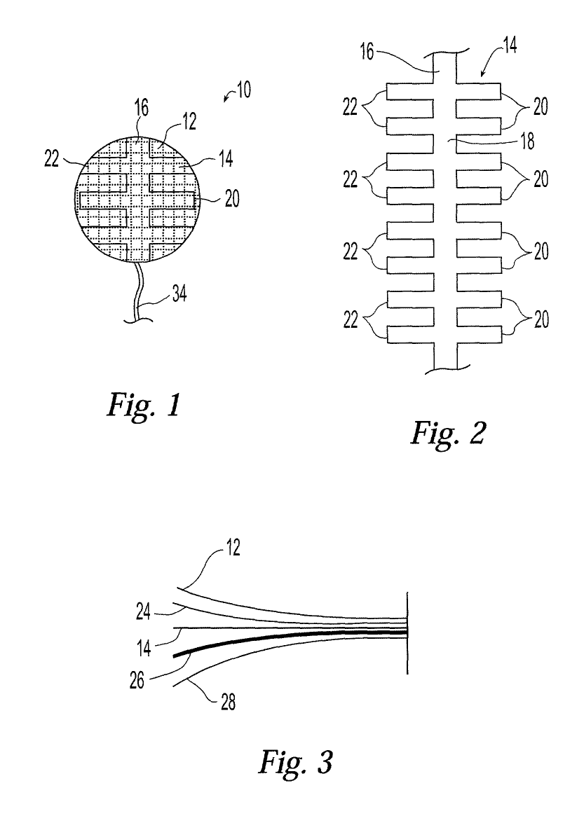

As shown in FIGS. 1 and 6, a flexible medical electrode 10 comprises a laminate of layers of a translucent, open mesh backing material 12, an intermediate layer 14 formed of ink-flooded, carbon coated, conductive vinyl sheet material. The conductive layer 14 is formed initially in an elongated strip 16, as shown in FIG. 2. Strip 16 is formed by die-cutting a cruciform shape out of an elongated strip of ink-flooded, carbon coated material. Strip 16 comprises a central stem 18 and a plurality of spaced opposed branches or furcations 20 and 22.

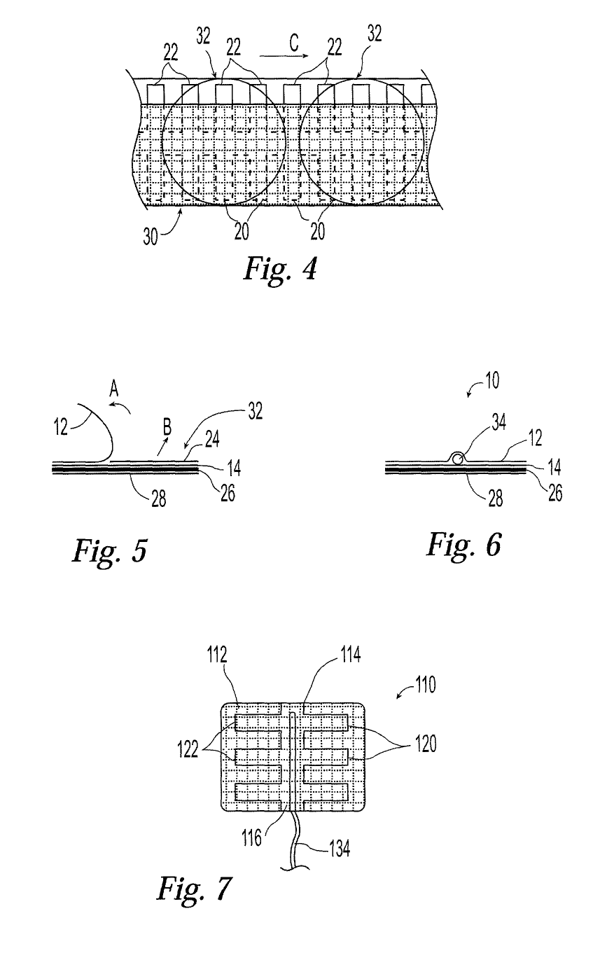

As shown in FIG. 3, electrode 10 is initially formed as a laminate of endless strips of backing material 12, which has an inner adhesive coating, partially covered by an opaque liner layer 24. Next comes the furcated conductive layer 14, a thick strip of conductive hydrogel 26, covered by a strip of carrier paper 28, forming a preliminary laminated strip 30, shown in FIG. 4. As seen in FIG. 4, all layers 12, 14, 26 and 28 are full width, while liner 24 is only about ¾ width.

These strips are pressed together so that the upper edge of backing strip is bonded to the outer edges of furcations 22, while liner strip 24 prevents adhesion of backing material 12 to the remainder of conductive strip 14. The sticky hydrogel strip 28 adheres to the underside of conductive layer 14 and backing material 12 between the voids between furcations 20, 22 and stem 18, and to the top of backing layer 28.

Strip 30 is conveyed in direction C along a production line to a stamping station where circular blanks 32 of partial laminate are formed by cutting through layers 12, 24, 14 and 26, leaving circular blanks as shown in FIG. 4. The scrap material formed by unwanted layers 12, 24, 14 and 26 are removed, leaving discrete blanks 32, as shown in FIG. 5. Next, the bottom edge of backing layer 12 is pulled up off of liner 24 is shown by arrow A in FIG. 5, and liner layer 24 is then removed from the blank 32 in the direction of arrow B. Next a bare wire electrode 34 is laid lengthwise of the direction of travel of blank 32 along the production line. Finally, another circular die cuts carrier strip 28, producing the discrete flexible medical electrodes 10, which comprises a laminate of backing material 12, conductive layer 14, bare wire electrode 34 and hydrogel 26, all on a removable carrier paper 12.

Electrodes 10 may then be stored until application to the human body is desired. Then, the electrode is stripped off backing paper layer 28 and applied to the skin in a well-known manner, with the hydrogel layer 26 contacting the skin and electrode 34 attached to a source of therapeutic electrical current. Because of the thinness and furcated configuration of conductive layer 14, the flexibility of woven backing layer 12, and the amorphous nature of the hydrogel layer 28, electrode 10 is extremely flexible and can readily conform to contours of the body.

One problem of prior art electrodes is the creasing and breaking of the continuity of the conductive layer. Such breaks are poorly accommodated by the conductivity of the hydrogel layer. However, this disperses and dilutes the electric current since conductivity is poor. In electrodes which use a printed ink conductor, any break in the conductive ink (quite common in stressed positions), results in degraded electrode performance. This is also true where carbon coated vinyl conductive layer alone is used. This problem is alleviated by the instant electrode by coating a carbon coated vinyl conductor with conductive ink, thus enabling the conductive ink to bridge any cracks in the carbon coated vinyl and provide good conductivity.

Another embodiment of electrode 10 is illustrated in FIG. 7, where an electrode 110 has a rectangular shape, comprising the same materials featuring conductive layer 114 comprising furcations 120, 122 extending from a stem 116, covered by cover layer 112 and having bare wire electrode 134. This embodiment is additionally advantageous because all furcations 120, 122 do not extend to the edge of electrode 110. This eliminates the problem of “edge bite”, which occurs when current escapes from the edge of the electrode directly to the skin, a common problem. This embodiment of flexible medical electrode 132 is made by the same process described above.

While only a preferred embodiment has been described and shown, obvious modifications are contemplated within the scope of this invention as defined by the following claims.

I claim:

1. A flexible laminated medical electrode for use on a human body part, comprising
a first backing layer of flexible, non-conductive material,
a second conductive layer comprising a sheet of carbon-loaded vinyl covered with a conductive ink and formed with a plurality of furcations extending from a central spine that includes means for connecting the electrode to a power source, and
a third hydrogel layer,
thereby providing sufficient flexure for the electrode to enable conformance to the contours of the body parts without adversely affecting electrical conductivity throughout the electrode.

2. The flexible laminated medical electrode of claim 1, wherein the second conductive layer has a cruciform shape.

3. The flexible laminated medical electrode of claim 1, wherein at least four second conductive layer furcations extend perpendicularly from opposite sides of the central spine.

4. The flexible laminated medical electrode of claim 1, including an adhesive that secures the first backing layer to the second conductive layer, and a lead wire, secured to the second conductive layer in engagement with the conductive layer spine by the first backing layer, for connecting the electrode to a power source.

5. The flexible laminated medical electrode of claim 4, wherein the lead wire extends along the length of the conductive layer spine.

6. The flexible laminated medical electrode of claim 1, wherein the laminated medical electrode has a periphery, and the furcations each extend from the spine to a position spaced from said periphery.

7. A method of making a flexible laminated medical electrode, comprising the steps of
providing an elongated sheet of conductive film that is flood-coated with conductive ink,
cutting said sheet to provide a continuous shape having a plurality of furcations extending from a central spine,
providing an elongated sheet of hydrogel on a backing liner,
providing an elongated sheet of flexible, non-conductive material having an adhesive coating on an inner side that is partially covered with a release liner so as to leave an exposed adhesive strip along one edge,
inserting said conductive film sheet between the sheet of hydrogel and the inner side of the sheet of non-conductive material to form an elongated sheet of partial laminate, with the adhesive strip securing a portion of the non-conductive material to the conductive film,
cutting the sheet of partial laminate into discrete partially laminated electrodes,
folding back the liner-covered portion of non-conductive material of each electrode to expose said central spine,
removing the release liner,
applying a lead wire along the central spine, and
engaging the non-conductive material with the conductive film sheet to secure the lead wire to the central spine and thereby form said flexible laminated medical electrode.
